(19) United States
(12) Patent Application Publication
(10) Pub. No.: US 2021/0161890 A1
(43) Pub. Date: Jun. 3, 2021

(54) COMPOSITIONS AND METHODS FOR TREATING BRAIN DYSFUNCTION

(71) Applicant: BACH PHARMA, INC., North Andover, MA (US)

(72) Inventors: Mark O. Henry, North Andover, MA (US); William S. Lynn, Hillsborough, NC (US)

(21) Appl. No.: 16/067,542

(22) PCT Filed: Dec. 31, 2016
(86) PCT No.: PCT/US2016/069636

§ 371 Date: Jun. 29, 2018

Related U.S. Application Data
(60) Provisional application No. 62/273,913, filed on Dec. 31, 2015.

(51) Int. Cl. A61K 31/502; A61K 9/00; A61P 25/28; A61P 25/22

(52) U.S. Cl. CPC A61K 31/502; A61P 25/22; A61P 25/28; A61K 9/0053

(57) ABSTRACT

The present invention relates to compositions that can be used, for example, in methods of treating medical conditions and symptoms associated with brain dysfunction, including but not limited to Gulf War illness (GWI), multiple chemical sensitivity (MCS), cognitive dysfunction (CD), multiple sclerosis (MS), and neurological disorders such as amyotrophic lateral sclerosis (ALS). in various embodiments, the compositions of the invention, which can be administered or prepared as a medicament for use in the treatment methods described herein, are phthalazinediones or pharmaceutically acceptable salts thereof. As described further below, the phthalazinedione can be 5-amino-2,3-dihydro-1;4-phthala2:inedione; an analog or variant thereof; or a salt of the specified compound, the analog, or the variant. The compounds described herein can be formulated as diagnostic or pharmaceutical compositions, and the invention features kits including one or more of these compounds.

### CROSS-REFERENCE TO RELATED APPLICATIONS

This application claims the benefit of the filing date of U.S. provisional application No. 62/273,913, filed Dec. 31, 2015, the entire content of which is hereby incorporated by reference in its entirety.

### STATEMENT REGARDING FEDERALLY SPONSORED RESEARCH

This invention was made with government support under grant number W81XWH-14-1-0572, awarded by the United States Army Medical Research and Material Command. The government has certain rights in the invention.

### FIELD OF THE INVENTION

The present invention relates to compositions that can be used, for example, in methods of treating medical conditions and symptoms associated with brain dysfunction, including but not limited to Gulf War Illness (GWI), multiple chemical sensitivity (MCS), cognitive dysfunction (CD), multiple sclerosis (MS), and neurological disorders such as amyotrophic lateral sclerosis (ALS). In various embodiments, the compositions of the invention, which can be administered or prepared as a medicament for use in the treatment methods described herein, are phthalazinediones or pharmaceutically acceptable salts thereof. As described further below, the phthalazinedione can be 5-amino-2,3-dihydro-1,4-phthalazinedione; an analog or variant thereof; or a salt of the specified compound, the analog, or the variant. The compounds described herein can be formulated as diagnostic or pharmaceutical compositions, and the invention features kits including one or more of these compounds.

### SUMMARY OF THE INVENTION

In a first aspect, the present invention features methods for treating a patient Who is suffering from a medical condition associated with brain dysfunction (e.g., GWI, ALS, MCS, CD, or any of the neurological disorders described herein) or any disease, condition, or syndrome that manifests as ataxia or in which ataxia is a prominent sign. We use the term “ataxia” as it is conventionally understood; ataxia is the loss of full control of one's bodily movements. While we may describe numerous embodiments of the invention as methods of treatment, it is to be understood that any of these embodiments can be presented, instead, in terms of the “use” of a composition as described herein. For example, the invention encompasses a method of treating ataxia with 5-amino-2,3-dihydro-1,4-phthalazinedione or a salt thereof (e.g., a monosodium salt thereof), and it likewise encompasses 5-amino-2,3-dihydro-1,4-phthalazinedione or a salt thereof (e.g., a monosodium salt thereof) for use in the treatment of ataxia. While we tend to use the term “5-amino-2,3-dihydro-14-phthalazinedione,” which is used commonly in the scientific and industrial literature, it is to be understood (and would be recognized by one of ordinary skill in the art), that the compound described by that term may also be described as 5-amino-2,3-dihydrophthalazine-1,4-dione. The latter descriptor was used in the provisional application from which this application claims the benefit of priority and may be the preferred IUPAC name. In case of doubt, we use the terms“5-amino-2,3-dihydro-1,4-phthalazinedione” and “5-amino-2,3-dihydrophthalazine-1,4-dione” interchangeably.

In one embodiment, the invention features methods of treating a patient by administering to the patient a therapeutically effective amount of a composition comprising an agent that conforms to the following structure:

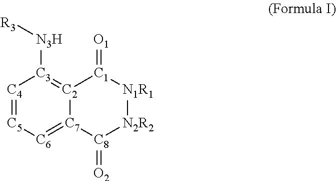

wherein, R₁ and R₂ are each, independently, hydrogen (H), lithium (Li), sodium (Na), potassium (K), rubidium (Rb), caesium (Cs), or francium (Fr);

R₃ is an alkyl, alkenyl, alkynly, aryl, alkoxyl, alkenyloxy, alkynyloxy, aryloxy, alkoxy carbonyl, alkylamino, alkylthio, alkylsullbnyl, or alkylsulfinyl, each optionally substituted with an alkyl, halogen, alkoxy, aryl or heteroaryl moiety:

C₁, C₂, C₃, C₄, C₅, C₆, C₇ and C₈ are each, independently, carbon 12 (¹²C) or an isotope of ¹²C (e.g., ¹³C);

N₁, N₂ and N₃ are each, independently, nitrogen 14 (¹⁴N) or an isotope of ¹⁴N (e.g., ¹⁵N); and O₁ and O₂ are each, independently oxygen 16 (¹⁶O) or an isotope of ¹⁶O (e.g., ¹⁷O or ¹⁸O).

The method can be employed to treat a medical condition such as a mood disorder or mood dysfunction (e.g., bipolar disorder, depression, or schizophrenia); a memory disorder or memory dysfunction (e.g., as occurs with amnesia, Alzheimer's disease, dementia, or Huntington's disease); anxiety or a stress-related condition (e.g., a post-traumatic stress disorder); an acute or chronic brain injury, including an acute or chronic brain injury caused by mitochondrial dysfunction or dysfunction of an endogenous retrovirus (e.g., a HERV (human endogenous retrovirus) such as Herv-K); Gulf War Illness (GWI; as defined by the United States Veterans' Administration and also known as Gulf War Veterans' Illness or Gulf War Syndrome); acute or chronic fatigue, which may or may not occur in the context of GWI; and/or ALS. The mood disorder or mood dysfunction may also occur in connection with cancer, another chronic illness, infection, or substance abuse. Accordingly, and in case of any doubt, the invention features methods of treating a mood disorder or mood dysfunction in a patient who is exhibiting the signs and symptoms of such a disorder or dysfunction and who has been diagnosed with and/or who is undergoing treatment for cancer, another chronic illness, infection or substance abuse. As indicated above, the invention also encompasses a compound of Formula I “for use” in the treatment of a mood disorder or mood dysfunction, or any of the conditions listed above or elsewhere herein.

Any of the methods described herein can include a step of identifying a patient in need of treatment. For example, a patient suspected of suffering from Gulf War Illness (GWI) may be subjected to a battery of tests for physical and mental function, as determined by the United States Veterans' Association. The tests may include assessment of neurological function, levels of fatigue (e.g., autonomic fatigue) and immune system function, genetic testing for susceptibility to GWI, behavioral tests, and assessment of quality of life (e.g., appetite, social interactions, sleep quality, and the like).

While the invention is not limited to compositions that achieve a positive treatment outcome by any particular physiological mechanism, we believe the compositions described herein promote hippocampal neurogenesis, and the methods of the invention and uses of the present compositions accordingly include methods of promoting neurogenesis in the hippocampus and compositions for use in promoting neurogenesis in the hippocampus.

By “treating” we mean administering a composition as described herein to a patient with an expectation that the patient will experience an improvement in the unwanted signs and/or symptoms of a condition as described herein (e.g., a less severe or less prolonged manifestation of the sign or symptom). A “therapeutically effective amount” of a composition is the amount that, upon administration, brings about such an improvement in a given patient or, more generally, results in improvement on average in a population of patients. As will be understood in the art, many of the medical conditions described herein are recognized by a prominent sign or symptom. For example, Alzheimer's disease is firmly associated with cognitive decline, multiple sclerosis, ALS, and Parkinson's disease are firmly associated with ataxia, post-traumatic stress disorders are firmly associated with anxiety, and so on. Accordingly, the present methods and uses of the compositions of the invention may be described equally well by reference to the condition itself or to a prominent sign or symptom thereof. For example, the methods of the invention encompass treating a patient who has Alzheimer's disease as well as treating a patient who is experiencing cognitive decline.

Other aspects and embodiments of the invention are described further below.

### BRIEF DESCRIPTION Of THE DRAWINGS

FIG. 1A is a flow diagram illustrating the sequence of trials, duration of trials, intervals between trials, examples of object types and floor patterns involved in a Pattern Separation Test (PST) according to one embodiment of the invention.

FIGS. 1B-1G are comparison charts illustrating the results of the Pattern Separation Test (PSI) in FIG. 1A.

FIGS. 2A-2F are comparison charts illustrating the results of a Sucrose Preference Test (SPT) according to one embodiment of the invention.

FIGS. 3A-3D are comparison charts illustrating that 5-amino-2,3-dihydro-1,4-phthalazittedione, monosodium salt treatment normalizes the expression of oxidative stress response genes prdx6, sod2, sqstm1 and srxn1 in GWI rats according to one embodiment of the invention.

FIG. 4 is a clustergram showing the expression of oxidative stress response genes in various animal groups according to one embodiment of the invention.

### DETAILED DESCRIPTION

As described further herein, the invention features, inter alia, compositions and methods for diagnosing and treating patients who are suffering from a medical condition associated with brain dysfunction, including those conditions that manifest with ataxia. The composition can be a compound that conforms to the following. Formula (I) or a pharmaceutical composition or formulation containing a compound that conforms to. Formula (I):

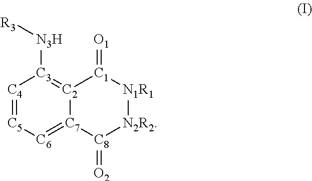

Within. Formula (I) R₁ and R₂ can each be, independently, hydrogen (H), lithium (Li), sodium (Na), potassium (K), rubidium (Rb), caesium (Cs), or francium (Fr); R₃ can be an alkyl, alkenyl, alkynly, aryl, alkoxyl, alkenyloxy, alkynyloxy, aryloxy, alkoxy carbonyl, alkylamino, alkylthio, alkylsulfonyl, or alkylsulfinyl, each optionally substituted with an alkyl, halogen, alkoxy, aryl or heteroaryl moiety; C₁, C₂, C₃, C₄, C₅, C₆, C₇, and C₈ can each be, independently, carbon 12 (¹²C) ) or an isotope of ¹²C (e.g., ¹³C¹); N₁, N₂ and N₃ can each be, independently, nitrogen 14 (¹⁴N) or an isotope of ¹⁴(e.g., ¹⁵N); O₁ and O₂O can each be, independently, oxygen 16 (¹⁶O) or an isotope of ¹⁶O (e.g., ¹⁷O or ¹⁸O). It is to be understood that the methods, uses, and compositions described herein can include either or both of a compound that conforms to Formula I or any other salt thereof (e.g., a pharmaceutically acceptable salt thereof).

In some embodiments (e.g., where the invention is the compound per se), the composition is a compound that. conforms to. Formula (I) with the proviso that when each of N₁-N₃ are ¹⁴N and each of O₁ and O₂ are ¹⁶O, then each and every one of C₂-C₇ cannot be C¹³. For example, the composition can be a compound that conforms to the following. Formula (II):

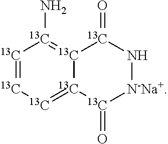

In other embodiments, the composition is a compound that conforms to. Formula (I) with the proviso that the compound is not N-ethanoyl-5-amino-2,3-dihydrophthalazine-1,4-dione (also known as 5-(acetoxyamino)-2,3-dihydrophthalazine-1,4-dione) or a salt thereof. For example, in some embodiments, the compound does not conform to following. Formula (III)

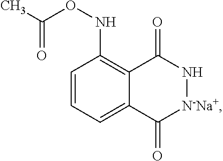

5-(hexanoyl oxyamino)-2,3-dihydrophthalazine-1,4-dione (also known as monosodium luminol henonyl) or a salt thereof. For example, in some embodiments, the compound does not conform to following Formula (IV):

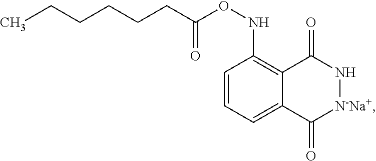

or N-methyl-5-amino-2,3-dihydrophthalazine-1,4-dione (also known as 5-(methylamino)-2,3-dihydrophthalazine-1,4-dione) or a salt thereof. For example, in some embodiments, the compound does not conform to following Formula (V):

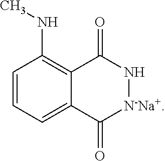

These compounds may also be excluded from one or more of the formulations, kits, or other compositions of matter described herein. Similarly, these compositions may be excluded from the prophylactic, diagnostic, or treatment methods described herein.

In some embodiments, the compound is a sodium salt of 5-amino-2,3-dihydro-1,4-phthalazinedione (also described as 5-amino-2,3-dihydrophthalazine-1,4-dione (luminol)) as shown in following. Formula (VI):

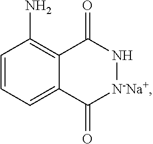

6-amino-2,3-dihydrophthalazine-1,4-dione, 5-amino-2,3-dihydrophthalazine-1,4-dion=8-yl (luminyl), N-bromo-5-amino-2,3-dihydrophthalazine-1,4-dione, N-chloro-5-amino-2,3-dihydrophthalazine-1,4-dione, N-fluoro-5-amino-2,3-dihydrophthalazine-1,4-dione, N-iodo-5-amino-2,3-dihydrophthalazine-1,4-dione, N-methyl-5-amino-2,3-dihydrophthalazine-1,4-dione, N-ethyl-5-amino-2,3-dihydrophthalazine-1,4-dione, N-propyl-5-amino-2,3-dihydrophthalazine-1,4-dione, N-isopropyl-5-amino-2,3-dihydrophthalazine-1,4-dione, N-methanoyI-5-amino-2,3-dihydrophthalazine-1,4-dione, N-ethanoyI-5-amino-2,3-dihydrophthalazine-1,4-dione N-propanoyl-5-amino-2,3-dihydrophthalazine-1,4-dione, N-hydroxyl-5-amino-2,3-dihydrophthalazine-1,4-dione, N-carboxyl-5-amino-2,3-dihydrophthalazine-1,4-dione, N-methanoyl-5-amino-2,3-dihydrophthalazine-1,4-dione, N-ethanol-5-amino-2,3-dihydrophthalazine-1,4-dione, N-propanol-5-amino-2,3-dihydrophthalazine-1,4-dione, N-methenyl-5-amino-2,3-dihydrophihalazine-1,4-dione, N-ethenyl-5-amino-2,3-dihydrophthalazine-1,4-dione, N-propenyl-5-amino-2,3-dihydrophthalazine-1,4-dione, N-methoxy-5-amino-2,3-dihydrophthalazine-1,4-dione, N-ethoxy-5-amino-2,3-dihydrophthalazine-1,4-dione, N-propoxy-5-amino-2,3-dihydrophthalazine-1,4-dione, N,N-dimethyl-5-amino-2,3-dihydrophthalazine-1,4-dione, N-acetylcysteine-5-amino-2,3-dihydrophthalazine-1,4-dione, N-acetylglutathione-5-amino-2,3-dihydrophthalazine-1,4-dione, 5-(hexanoyl oxyamino)-2,3-dihydrophthalazine-1,4-dione, 5-(methylamino)-2,3-dihydrophthalazine-1,4-dione, or 5-(acetoxyamino)-2,3-dihydrophthalazine-1,4-dione, a pharmaceutically acceptable salt. thereof (i.e., of any of the foregoing), or an isotopic derivative thereof (i.e., an isotopic derivative of any of the foregoing). For example, the agent can be 5-amino-2,3-dihydro-1,4-phthalazinedione, monosodium salt, also known as 3-aminophthalydrazide, sodium salt, 3-aminophthalic hydrazide, sodium salt, 0-aminophthaloyl hydrazine, sodium salt, to merit, or tamerite, or trademarked as MSL® and/or GVT®. In certain embodiments, the agent can be monosodium 5-amino-23-dihydro-1,4-phthalazinedione (as of Formula (VI)), monosodium 5-methylamino-23-dihydro-1,4-phthalazinedione (as of Formula (V) , monosodium 5-acetoxyamino-2,3-dihydro-1,4-phthalazinedione (as of Formula (III)), or monosodium 5-(hexanoyl oxylamino)-2,3-dihydro-1,4-phthalazinedione (as of Formula (IV)). The diagnostic standards, pharmaceutical compositions, and kits of the invention can include one or more of (i.e., any combination of) the compounds described herein. By diagnostic standards, we mean formulations generated to serve as reference standards in a diagnostic or other assay conducted using mass spectrometry.

Patients amenable to treatment: As described above, a medical condition treated by the pharmaceutical compositions described herein can be, or can include as a prominent symptom, lack of muscle control and, as a result, loss of full control over bodily movements (ataxia), including lack of movement or of proper, controlled movement of the eye(s), difficulty walking or running in a normal, controlled manner (as can be detected and assessed by gait analysis), lack of coordination (as can be detected and assessed by any test for manual dexterity), aphasia, apraxia, or asthenia. A patient experiencing ataxia may have ALS, MS, or Parkinson's disease. The medical condition can also be, or can include as a prominent symptom, fatigue, Tourette's syndrome, narcolepsy, back and/or neck pain, and headaches (including migraine headaches (e.g., visual migraines), cluster headaches, and tension headaches). In some embodiments, the patient can exhibit signs of neuronal fatigue, in some embodiments, the patient can have multiple sclerosis. In some embodiments, the patient can have a multiple chemical sensitivity (MCS) which can, in turn, include a range of symptoms that may be attributed to exposure to chemicals that are commonly used in building materials, industrial sites, and battle grounds. MCS has also been referred to as environmental illness, sick building syndrome, and idiopathic environmental intolerance. Where a patient is experiencing fatigue, the step of diagnosing the patient and/or monitoring the patient's treatment can include determining the rate of mitochondrial recovery after a defined exertion (e.g., after exercise in a sample from the patient that includes, for example, white blood cells). The rate of mitochondrial recovery or any other assessment of a patient can be compared to a reference standard defining a healthy/normal/desired level. In other embodiments, the patient can be suffering from a mood. disorder or mood dysfunction (e.g., bipolar disorder, depression, or schizophrenia); a memory disorder or memory dysfunction (e.g., as occurs with amnesia, Alzheimer's disease, dementia, or Huntington's disease); anxiety or a stress-related condition (e.g., a post-traumatic stress disorder); an acute or chronic brain injury, including an acute or chronic brain injury caused by mitochondrial, dysfunction or an endogenous retrovirus; Gulf War Illness (GWI; as defined by the United States Veterans' Administration and also known as Gulf War Veterans' Illness or Gulf War Syndrome); acute or chronic fatigue, which may or may not occur in the context of GWI; and or ALS. Any of the methods of the invention can include a step of assessing a patient's weight, and in some embodiments, patients who are obese or who have been diagnosed as having obesity-induced inflammation can be excluded from treatment. In other embodiments, the excluded patient can have a metabolic syndrome or a medical condition associated with heavy metal intoxication. In some embodiments, the mood disorder or mood dysfunction can be bipolar disorder, depression, schizophrenia, or can be associated with cancer, another chronic illness, infection, or substance abuse. The anxiety or stress-related disorder can be a post-traumatic stress disorder. The memory disorder or memory dysfunction can be amnesia, or is associated with a diagnosis of Alzheimer's disease, dementia, Huntington's disease, or Parkinson's disease.

As noted, in some embodiments, the medical condition can be associated with Gulf War Illnes (GWI) or can, more generally, be a cognitive impairment associated with mitochondrial dysfunction or with other neuronal stresses, particularly those that impair neurogenesis in the hippocampal region of the brain. Thus, the pharmaceutical compositions described herein can be administered to treat cognitive impairment and/or to facilitate cognitive rehabilitation. While the present methods are intended to apply to GWI, the invention is not so limited. The patient can be one who has experienced baffle or worked in a battle-torn area, regardless of the precise time or place. Further, patients who have experienced the stress of preparing for military service are amenable to treatment, as are patients who support others who are preparing for service or recovering from a deployment (e.g., family members or close friends). The conditions amenable to treatment can manifest in various ways, and any given patient can be suffering front a variety of symptoms including, but not limited to, fatigue chronic fatigue syndrome), with mitochondrial dysfunction headache, memory problems, muscle or joint pain, muscle weakness (ataxia), diarrhea, dyspepsia, indigestion, or other gastrointestinal problems, other neurological problems, tumors or blood cancers, skin conditions, arthritis, or respiratory problems. In one embodiment, the patient has been diagnosed as having a post-traumatic stress disorder (PTSD), a chronic, multi-symptom illness, or a combination thereof.

As described above, the medical condition being treated by the present compositions may be caused by physical and psychological issues involving any war zone deployment (e.g., deployment during the Gulf War) or may have been caused by exposure to one or more hazards (e.g., toxins). In other words, the medical condition may have been precipitated by chemical or environmental factors, particularly in the context of experiencing or preparing for a stressful situation such as military deployment. The hazard may be, but is not limited to, exposure to a nerve gas such as sarin, a pyridostigmine bromide pill, a depleted uranium munition, an anthrax vaccine, a botulinum vaccine, oil, smoke from burning oil, pesticides, or microwaves. The hazard may also be exposure to radiation (e.g., electromagnetic radiation).

In some embodiments, the medical condition may be associated with one or more neurological disorders, and these neurological disorders may, in turn, be precipitated by preparing for, living or working within, or attempting to cope with a stressful and/or chemical-laden environment. For example, the neurological disorder may be associated with coping mechanisms that include addiction (e.g., alcohol or substance abuse). As noted above, many of these disorders manifest in ataxia, and any of the methods described herein can include a step of assessing a patient for ataxia prior to treatment.

Although the invention was developed with human patients in mind, it is not so limited. The present methods can be carried out for the benefit of any vertebrate animal, including domesticated mammals (e.g., dogs and cats) and birds kept as pets or in zoos. The subject can also be an animal kept as livestock (e.g., cattle, sheep, chickens, horses, pigs, or goats). in other embodiments, the present compositions can be applied to a cell, tissue, organ, organ system, organism, or a medium containing one or more of these (e.g., in a laboratory or cell culture).

Formulations and dosages: As described above, a composition of the invention can be administered to the patient orally, topically, by inhalation, nasal delivery (at, for example, 50 to 100 mg/ml administered. 5 or 6 times per day, for a daily dose of about 25-1,000 mg, for about 20 days) to the brain or by an injection. Our work to date suggests that administration to the nasal passages, buccal mucosal (at, e.g., about 200 to 500 mg daily for about 20 days) and/or sublingual tissue (at, e.g., about 25 mg to 50 mg four or five times per day) may be preferred. The compositions intended for pharmaceutical use can also be formulated for auricular administration (to or by way of the ear), conjunctival administration (to or by way of the conjunctiva), a cutaneous administration to the skin, dental or intracoronal administration (administration to a tooth or teeth, including to the portion of a tooth covered by enamel), an electro-osmosis administration (e.g., administration through the diffusion of a substance through a membrane in an electric field), an endocervical administration (e.g., administration within the canal of the cervix uteri), an endosinusial administration (e.g., administration within the nasal sinuses of the head), an endotracheal administration (e.g., administration directly into the trachea), an enteral administration (e.g., administration directly into the intestines), an epidural administration (e.g., administration upon or over the dura mater), an extracorporeal administration (e.g., administration outside of the body), a hemodialysis (e.g., administration through hemodialysate fluid), an infiltration (e.g., administration that results in substances passing into tissue spaces or into cells), an interstitial administration (e.g., administration to or in the interstices of a tissue), an intra-abdominal administration (e.g., administration within the abdomen), an intra-arterial administration (e.g., administration within an artery or arteries), an intra-articular administration (e.g., administration within a joint), an intrabiliary administration (e.g., administration within the bile, bile ducts or gallbladder), an intrabronchial, administration (e.g., administration within a bronchus), an intrabursal administration (e.g., administration within a bursa), an intracardiac administration (e.g. administration with the heart), an intracartilaginous administration (e.g., administration within a cartilage), an intracaudal administration (e.g., administration within the cauda equine), an intracavernous administration (e.g., administration within a pathologic cavity, such as occurs in the lung in tuberculosis), an intracavitary administration (e.g., administration within a non-pathologic cavity, such as that of the cervix, uterus, or penis, or such as that which is formed as the result of a wound), an intracerebral administration, (e.g., administration within the cerebrum), an intracisternal administration (e.g., administration within the cisterna magna cerebellomedularis), tan intracorneal administration (e.g., administration within the cornea), an intracoronary administration (e.g., administration within the coronary arteries), an intracorporus cavemosum e.g., administration within the dilatable spaces of the corporus cavernosa of the penis), tin ntradermal administration (e.g., administration within the dermis), an intradiscal administration (e.g., administration within a disc), an intraductal administration (e.g., administration within the duct of a gland), an intraduodenal administration (e.g., administration within the duodenum), an intradural administration (e.g., administration within or beneath the dura), an intraepidermal administration (e.g., administration within the epidermis), an intraesophageal administration (e.g., administration within the esophagus), an intragastric administration (e.g., administration within the stomach), an intragingival administration (e.g., administration within the gingivae), an intraileal administration, an intralesional administration (e.g., administration within or introduced directly into a localized lesion), an intraluminal administration (e.g., administration within the lumen of a tube), an intralymphatic administration (e.g., administration within the lymph), an intramedullary administration (e.g., administration within the marrow cavity of a bone), an intrameningeal administration (e.g., administration within the meninges), an intramuscular administration, an intraocular administration (e.g., administration within the eye), an intrapericardial administration (e.g., administration within the pericardium), an intraperitoneal administration, an intrapleural administration (e.g., administration within the pleura), an intraprostatic administration (e.g., administration within the prostate gland), an intrapulmonary administration (e.g., administration within the lungs or its bronchi), an intrasinal administration (e.g., administration within the nasal or periorbital sinuses), an intraspinal administration (e.g., administration within the vertebral column), an intrasynovial administration (e.g., administration within the synovial cavity of a joint), am intratendinous administration (e.g., administration within a tendon), an intratesticular administration (e.g., administration within the testicle), an intrathecal administration (e.g., administration within the cerebrospinal fluid at any level of the cerebrospinal axis, including injection into the cerebral ventricles), an intrathoracic administration (e.g., administration within the thorax), an intratubular administration (e.g., administration within the tubules of an organ), an intratumor administration (e.g., administration within a tumor), an intratympanic administration (e.g., administration within the aunts media), an intrauterine administration (e.g., administration within the uterus), an intravascular administration (e.g., administration within a vessel or vessels), an intravenous administration (e.g., administration within or into a vein or veins), an intravenous bolus administration (e.g., administration within or into a vein or veins all at once), an intravenous drip administration (e.g., administration within or into a vein or veins over a sustained period of time), an intravesical administration (e.g., administration within the bladder), an intravitreal administration (e.g., administration within the vitreous body of the eye), an iontophoresis (e.g., administration by means of an electric current where ions of soluble salts migrate into the tissues of the body), an irrigation (e.g., administration to bathe or flush open wounds or body cavities), a laryngeal administration (e.g., administration directly upon the larynx), a nasal administration (e.g., administration to the nose; administered by way of the nose), a nasogastric administration (e.g., administration through the nose and into the stomach, usually by means of a tube), an occlusive dressing technique (e.g., administration by the topical route which is then covered by a dressing which occludes the area), an ophthalmic administration (e.g., administration to the external eye), an oral administration (e.g., administration to or by way of the mouth), an oropharyngeal administration (e.g., administration directly to the mouth and pharynx), a parenteral administration (e.g. administration by injection, infusion, or implantation), a percutaneous administration (e.g., administration through the skin), a periarticular administration (e.g., administration around a joint), a peridural administration (e.g., administration to the outside of the dura mater of the spinal cord), a perineural administration (e.g., administration surrounding a nerve or nerves), aperiodontal administration (e.g., administration around a tooth), a rectal administration (e.g., administration to the.rectum), a respiratory administration (e.g., administration within the respiratory tract by inhaling orally or nasally for local or systemic effect), a retrobulbar administration (e.g., administration behind the pons or behind the eyeball), soft tissue administration (e.g., administration into any soft tissue), a subarachnoid administration (e.g., administration beneath the arachnoid), a subconjunctival administration (e.g., administration beneath the conjunctiva), a subcutaneous administration e,g., administration beneath the skin), a sublingual administration (e.g., administration beneath the tongue), a submucosal administration (e.g., administration beneath the mucous membrane), a topical administration administration to a particular spot on the outer surface of the body), a transdermal administration (e.g., administration through the dermal layer of the skin to the systemic circulation by diffusion), a transmucosal administration (e.g., administration across the mucosa), a transplacental administration (e.g., administration through or across the placenta), a transtracheal administration (e.g., administration through the wall of the trachea), a transtympanic administration (e.g., administration across or through the tympanic cavity), an ureteral administration (e.g., administration into the ureter), an urethral administration (e.g., administration into be urethra), a vaginal administration (e.g., administration into the vagina), or a combination thereof.

In considering the formulations and dosages to be administered, one can invoke principles of personalized medicine, and such regimens are encompassed by the present methods. During the administration of the composition, a dose level can vary as a function of the one or more compositions, the severity of the symptoms and the susceptibility of the subject to side effects. Preferred dosages for a given composition are readily determinable by those of skill in the art by a variety of means. In many embodiments, multiple doses of one or more compositions are administered.

The pharmaceutical compositions may be administered one to three times per month, every other week (qow), 1-6 times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. For example, the composition can be administrated five times a week for eight weeks. In severe cases, the patient may require administration at one or more of these delivery doses for the remainder of his or her life.

In connection with the above-described methods, a composition as described herein may be administered to the patient at a dosage from about 10 mg/kg to 500 mg/kg patient bodyweight per day, in 1 to 5 divided doses per day. For example, the composition may be administered at a dosage of about 40 mg/kg, about 80 mg/kg, or about 160 mg/kg patient body weight per day. Where liquid or powdered formulations are prepared for administration to a mucous membrane (e.g., within the nose or mouth), the compound can be present at between about 50-100 mg/ml (e.g., 70 mg/ml). The term “about” is used herein to indicate that a value includes an inherent variation of error for the device or the method being employed to determine the value or plus-or-minus 10% of the value, whichever is greater.

In various embodiments, the composition can be formulated in the form of a pill, a capsule, a granule, a tablet, a pallet, a suspension, an injection, an infusion, a suppository, a continuous delivery system, a syrup, a tincture, an ointment, a cream, eye drops, eardrops, a fush, a lavage, a slow absorbing depot, a dressing, a lozenge, or any pharmaceutically acceptable application or as a nutritional supplement.

The compounds disclosed herein can be formulated with conventional carriers and excipients, which can be selected in accord with ordinary practice. Tablets can typically contain excipients, glidants, tillers, binders and the like. Aqueous formulations can be prepared in sterile form, and when intended for delivery by other than oral administration generally can be isotonic. Formulations can contain excipients (e.g., excipients set forth in the Handbook of Pharmaceutical Excipients, 5th Ed.; Rowe, Sheskey, and Owen, Eds.; American Pharmacists Association; Pharmaceutical Press: Washington, D.C., 2006).

Excipients can include ascorbic acid or other antioxidants, chelating agents such as EDTA, carbohydrates such as dextrin, hydroxyalkylcellulose, hydroxyalkylinethylcellulose, stearic acid or the like.

The pH of the formulations can range from about 3 to about 11, and can be about 7 to 10. While it is possible for the active ingredients to be administered alone it can be preferable to present them as pharmaceutical formulations. The formulations of the agent, as disclosed herein, can include at least one active ingredient, as above defined, together with one or more acceptable Garvie's thereof and optionally other therapeutic ingredients. The carrier(s) should be “acceptable” in the sense of being compatible with the other ingredients of the formulation and physiologically innocuous to the recipient thereof. The formulations can include those suitable for the foregoing administration routes. The formulations can conveniently he presented in unit dosage form and may be prepared by any of the methods well known in the art of pharmacy. Techniques and formulations generally can be found in Remington's Pharmaceutical Sciences, Mack Publishing Company, Easton, Pa., (1985). Such methods can include the step of bringing into association the active ingredient with the carrier that constitutes one or more accessory ingredients. In general the formulations can be prepared by uniformly and intimately bringing into association the active ingredient with liquid carriers, finely divided solid carriers, or both, and then, if necessary, shaping the product. Formulations of the presently disclosed subject matter suitable for oral administration can be presented as discrete units such as capsules, cachets, or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can also be administered as a bolus, electuary, or paste.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets can be made by molding in a suitable machine a mixture of the powdered active ingredient moistened with an inert liquid diluent. The tablets can be coated or scored and optionally are formulated so as to provide slow or controlled release of the active ingredient therefrom. For administration to the eye or other external tissues (e.g., mouth and skin), the formulations can be applied as a topical ointment or a cream containing the active ingredient(s). When formulated in an ointment, the active ingredients can be employed with either a paraffnic or a water-miscible ointment base. Alternatively, the active ingredients can be formulated in a cream with an oil-in-water cream base. If desired, the aqueous phase of the cream base can include at least 30% w/w of a polyhydric alcohol (e.g., an alcohol having two or more hydroxyl groups such as propylene glycol, butane 1,3-diol, mannitol, sorbitol, glycerol and polyethylene glycol (including PEG 400) and mixtures thereof). The topical formulations can include a compound (e.g., dimethyl sulphoxide or related analogs) that enhances absorption or penetration of the active ingredient through the skin or other affected areas.)

The oily phase of the emulsions of this disclosed subject matter can be constituted from known ingredients in a known manner. The phase can include merely an emulsifier (otherwise known as an emulgent) or a mixture of at least one emulsifier with a fat or an oil or a combination thereof Preferably, a hydrophilic emulsifier can be included together with a lipophilic emulsifier that acts as a stabilizer. It is also preferred to include both an oil and a fat. Together, the emulsifier(s) with or without stabilizer(s) can make up the so-called emulsifying wax, and the wax together with the oil and fat make up the so-called emulsifying ointment base that forms the oily dispersed phase of the cream formulations. Emulgents and emulsion stabilizers suitable for use in the formulation of the agent, as disclosed herein, can include TWEEN 60, SPAN 80, cetostearyl alcohol, benzyl alcohol, myristyl alcohol, glyceryl mono-stearate, or sodium lauryl sulfate.

The choice of suitable oils or fats for the formulation can be based on achieving the desired cosmetic properties. The cream can be a non-greasy, non-staining and washable product with suitable consistency to avoid leakage from tubes or other containers. Straight or branched chain, mono- or dibasic alkyl esters (e.g., diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters (Crodamol CAP)) may be used. These may be used alone or in combination depending on the properties required, Alternatively, high melting point lipids such as white soft paraffin and/or liquid paraffin or other mineral oils can be used.

Pharmaceutical formulations can include the agent together with one or more pharmaceutically acceptable carriers or excipients and optionally other therapeutic agents. Pharmaceutical formulations containing the active ingredient can be in any form suitable for the intended method of administration.

When used for oral use for example, tablets, troches, lozenges, aqueous or oil suspensions, dispersible powders or granules, emulsions, hard or soft capsules, syrups or elixirs can be prepared.

Compositions intended for oral use can be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient that is suitable for manufacture of tablets can be acceptable. These excipients can be inert diluents (e.g., calcium or sodium carbonate, lactose, lactose monohydrate, croscarmellose sodium, povidone, calcium or sodium phosphate), granulating and disintegrating agents (e.g., maize starch or alginic acid), binding agents (e.g., cellulose, microcrystalline cellulose, starch, gelatin or acacia), or lubricating, agents (e.g., magnesium stearate, stearic acid or talc). Tablets can be uncoated or coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be used.

Formulations for oral use can be also presented as hard gelatin capsules where the active ingredient, is mixed with an inert solid diluents (e.g., calcium phosphate or kaolin), or as soft gelatin capsules wherein the active ingredient is mixed with water or an oil medium (e.g., peanut oil, liquid paraffin, or olive oil).

Aqueous suspensions of the agent, as disclosed herein, can contain the active materials in admixture with excipients suitable for the manufacture of aqueous suspensions. Such excipients can include a suspending agent (e.g., sodium carboxymethylcellulose, methylcellulose, hydroxypropyl methylcelluose sodium alginate, polyvinylpyrrolidone, gum tragacanth or gum acacia), a dispersing or wetting agent such as a naturally occurring phosphatide (e.g., lecithin), a condensation product of an alkylene oxide with a thtty acid (e.g., polyoxyethylene stearate), a condensation product of ethylene oxide with a long chain aliphatic alcohol (e.g., heptadecaethyleneoxycetanol), or a condensation product of ethylene oxide with a partial ester derived from a fatty acid and a hexitol anhydride (e.g., polyoxyethylene sorbitan monooleate). The aqueous suspension can also contain one or more preservatives (e.g., ethyl or n-propyl hydroxy-benzoate), one or more coloring agents, one or more flavoring agents and one or more sweetening agents (e.g., sucrose or saccharin). Oil suspensions can be formulated by suspending the active ingredient in a vegetable oil (e.g., arachis oil, olive oil, sesame oil, or coconut oil), or in a mineral oil (e.g., liquid paraffin). The oral suspensions can contain a thickening agent (e.g., beeswax, hard paraffin, or cetyl alcohol). Sweetening agents and/or flavoring agents can be added to provide a palatable oral preparation. These compositions can be preserved by the addition of an antioxidant such as ascorbic acid.

Dispersible powders and granules of the agent, as disclosed herein, can be suitable for preparation of an aqueous suspension by the addition of water provide the active ingredient in admixture with a dispersing or wetting agent, a suspending agent, and one or more preservatives. Suitable dispersing or wetting agents and suspending agents can be exemplified by those disclosed above. Additional excipients (e.g., sweetening, flavoring and coloring agents) can also be present.

The pharmaceutical compositions of the agent, as disclosed herein, can also be in the form of oil-in-water emulsions. The oily phase can be a vegetable oil (e.g., olive oil or arachis oil), a mineral oil (e.g., liquid paraffin), or a mixture of these.

Formulations fir oral use can also be presented as hard gelatin capsules where the active ingredient is mixed with an inert solid diluent (e.g., calcium phosphate or kaolin), or as soft gelatin capsules wherein the active ingredient is mixed with water or an oil medium (e.g., peanut oil, liquid paraffin, or olive oil).

Aqueous suspensions of the agent, as disclosed herein, can contain the active materials in admixture with excipients suitable for the manufacture of aqueous suspensions. Such excipients can include a suspending agent (e.g., sodium carboxymethylcellulose, methylcellulose, hydroxypropyl mothylcelluose, sodium alginate, polyvinylpyrrolidone, gum tragacanth or gum acacia), a dispersing or wetting agent (e.g., a naturally occurring phosphatide (e.g., lecithin)), a condensation product of an alkylene oxide with a fatty acid (e.g., polyoxyethylene stearate), a condensation product of ethylene oxide with a long chain aliphatic alcohol (e.g., heptadecaethyletteoxycetanol), a condensation product of ethylene oxide with a partial ester derived from a fatty acid and a hexitol anhydride (e.g., polyoxyethylene sorbitan ruonooleate). The aqueous suspension can also contain one or more preservatives (e.g., ethyl or n-propyl p-hydroxy-benzoate), one or more coloring agents, one or more flavoring agents and one or more sweetening agents (e.g., sucrose or saccharin). Oil suspensions can be formulated by suspending the active ingredient in a vegetable oil, such as arachis oil, olive oil, sesame oil, or coconut oil, or in a mineral oil (e.g., liquid paraffin). The oral suspensions can contain a thickening agent (e.g., beeswax, hard paraffin, or cetyl alcohol). Sweetening agents or flavoring agents can be added to provide a palatable oral preparation. These compositions can be preserved by the addition of an antioxidant such as ascorbic acid.

Dispersible powders and granules of the agent, as disclosed herein, can be suitable for preparation of an aqueous suspension by the addition of water provide the active ingredient in admixture with a dispersing or wetting agent, a suspending agent, and one or more preservatives. Suitable dispersing or wetting agents and suspending agents can be exemplified by those disclosed above. Additional excipients (e.g., sweetening, flavoring and coloring agents) can also be present.

The pharmaceutical compositions of the agent, as disclosed herein, can also be in the form of oil-in-water emulsions. The oily phase can be a vegetable oil (e.g., olive oil or arachis oil), a mineral oil (e.g., liquid paraffin), or a mixture of these. Suitable emulsifying agents can include naturally occurring, gums (e.g., gum acacia and gum tragacanth), naturally occurring phosphatides (e.g., soybean lecithin), esters or partial esters derived from fatty acids and hexitol anhydrides (e.g., sorbitan monooleate), and condensation products of these partial esters with. ethylene oxide (e.g., polyoxyethylene sorbitan monooleate). The emulsion can also contain sweetening and flavoring agents. Syrups and elixirs can be formulated with sweetening agents (e.g., glycerol, sorbitol, or sucrose). Such formulations can also contain a demulcent, a preservative, a flavoring or a coloring agent.

The pharmaceutical compositions of the agent, as disclosed herein, can be in the form of a sterile injectable preparation, such as a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parenterally acceptable diluent or solvent (e.g., a solution in 1,3-butane-diol or prepared as a lyophilized powder).

Among the acceptable vehicles and solvents that can be employed are water, Ringer's solution and isotonic sodium chloride solution. In addition, sterile fixed oils can be conventionally employed as a solvent or suspending, medium. For this purpose any bland fixed oil can be employed (e.g., synthetic mono- or diglycerides). Fatty acids (e.g., oleic acid) can also be used in the preparation of injectables.

The amount of active ingredient that can be combined with the carrier material to produce a single dosage form should vary depending upon the host treated and the particular mode of administration. Far example, a time-release formulation intended for oral administration to humans may contain approximately 1 to 100 mg of active material compounded with an appropriate and convenient amount of carrier material that may vary from about 5 to about 95% of the total compositions (weight: weight).

The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye can include eye drops wherein the active ingredient is dissolved or suspended in a suitable carrier, especially an aqueous solvent for the active ingredient. The active ingredient can be present in such formulations in a concentration of 0.5 to 20%, 0.5 to 10%, or 1.5% to 8% (w/w).

Formulations suitable for topical administration in the mouth can include lozenges including the active ingredient in a flavored basis, typically sucrose and acacia or tragacantly, pastilles including the active ingredient in an inert basis such as gelatin and glycerin, or sucrose and acacia; and mouthwashes including the active ingredient in a suitable liquid carrier.

Formulations for rectal administration can be presented as a suppository with a suitable base including for example cocoa butter or a salicylate. Formulations suitable for intrapulmonary or nasal administration can have a particle size in the range 0.1 to 500 microns (including particle sizes in a range between 0.1 and 500 microns in increments microns such as 0.5, 1, 30 microns, 35 microns, etc.), which can be administered by rapid inhalation through the nasal passage or by inhalation through the mouth so as to reach the alveolar sacs. Suitable formulations can include aqueous or oily solutions of the active ingredient. Formulations suitable for aerosol or dry powder administration can be prepared according to conventional methods and may be delivered with other therapeutic agents such as compounds heretofore used in the treatment or prophylaxis of a given condition. Formulations suitable for vaginal administration can be presented as pessaries, tampons, creams, gels, pastes, foams or spray formulations containing in addition to the active ingredient such carriers as are known in the art to be appropriate.

Formulations suitable for parenteral administration can include aqueous and nonaqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents.

The formulations can be presented in unit-dose or. ulti-dose containers (e.g., sealed ampoules and vials) and can be stored in a freeze-dried (lyophilized) condition requiring the addition of the sterile liquid carrier (e.g., water) for injection, immediately prior to use. Extemporaneous injection solutions and suspensions can be prepared from sterile powders, granules and tablets of the kind previously described. Preferred unit dosage formulations can be those containing a daily dose or unit daily sub-dose, as herein above recited, or an appropriate fraction thereof, of the active ingredient.

In addition to the ingredients particularly mentioned above, the formulations of the agent, as disclosed herein, can include other agents conventional in the an having regard to the type of formulation in question (e.g., those suitable for oral administration can include flavoring agents).

The agent, as disclosed herein, can also be formulated to provide controlled release of the active ingredient to allow less frequent dosing, or to improve the pharmacokinetic or toxicity profile of the active ingredient. Accordingly, the agent, as disclosed herein, can also be provided in compositions including one or more agents formulated for sustained or controlled release.

An effective dose of active ingredient can depend at least on the nature of the condition being treated, toxicity, whether the compound is being used prophylactically (typically lower doses), the method of delivery, and the pharmaceutical formulation, and is determined by the clinician using conventional dose escalation studies. It can be expected to be from about 0.0001 to about 100 mg/kg body weight per day, typically, from about 0.01 to about 10 mg/kg body weight per day, more typically, from about 0.01 to about 5 mg/kg body weight per day, and more typically, from about 0.05 to about 0.5 mg/kg body weight per day.

If desired, the compounds of the presently disclosed subject matter can be applied in conjunction with one or more inert or inactive ingredients. The agent, as disclosed herein, can be administered by any route appropriate to the condition to be treated. Suitable routes can include oral, rectal, nasal, topical (including buccal and sublingual), vaginal and parenteral (including subcutaneous, intramuscular, intravenous, intradermal, intrathecal and epidural), and the like.

Diagnostic and Pharmaceutical Kits: As described further herein, the invention also features kits for aiding in the diagnosis and treatment of a patient who is suffering from a medical condition associated with brain dysfunction or as otherwise described herein. The kit may include a therapeutically effective amount of one of more of the compositions described above.

In some embodiment, the agent can be used in combination with other active ingredients. The combinations can be selected based on the condition to be treated, cross-reactivities of ingredients and pharmaco-properties of the combination. The agent can also be combined with one or more other active ingredients in a unitary dosage form liar simultaneous or sequential administration to a patient by the same or differem routes of administration. The combination therapy can be administered as a simultaneous or sequential regimen. When administered sequentially, the cotribination can be administered in two or more administrations.

In general, during alternation therapy, an effective dosage of each active ingredient can be administered sequentially (i.e., serially), whereas in combination therapy, effective dosages of two or more active ingredients can be administered together. The combination therapy may provide “synergy” and “synergistic effect” (i.e., the effect achieved when the active ingredients used together is greater than the sum of the effects that results from using the compounds separately). In certain embodiments, a synergistic effect can be attained when the active ingredients are: (1) co-formulated and administered or delivered simultaneously in a combined formulation; (2) delivered by alternation or in parallel as separate formulations; or (3) by some other regimen. In alternation therapy, the synergistic effect can also be attained when the compounds are administered or delivered sequentially (e.g., in separate tablets, pills, or capsules, or by different infections in separate syringes).

In some embodiments, pharmaceutical kits useful in the presently disclosed subject matter, which can include a therapeutically effective amount of a pharmaceutical composition including (a) a compound of component andior (b) one or more compounds of component, in one or more sterile containers, can also be within the ambit of the presently disclosed subject matter. Sterilization of the container can be carried out using conventional sterilization methodology well known to those skilled in the art. Component (a) and/or component (b) can be in the same sterile container or in separate sterile containers. The sterile containers or materials can include separate containers, or one or more multi-part containers, as desired. Component (a) and/or component (h) can be separate, or physically combined into a single dosage form or unit. The kits can further include one or more of various conventional pharmaceutical kit components (e.g., one or more pharmaceutically acceptable carriers, additional vials for mixing the components), as should be readily apparent to those skilled in the art. Instructions, either as inserts or as labels, indicating quantities of the components to be administered, guidelines for administration, and/or guidelines for mixing the components, can also be included in the kit.

Evaluation Method: The methods described herein may include a step to evaluate the effect of the composition (e.g., by assessing a cognitive function or mood of the patient). For example, the methods can include a Pattern Separation Test PST) for assessing cognitive function, a Sucrose Preference Test (SPI) for assessing mood function, or both.

Alternatively or in addition, the evaluation step can include evaluating an oxidative stress response or determining the expression level of one or more antioxidant genes in the patient. The gene may be prdx6, sod-1, sod2, sgstm1, srsn1, a cat gene (encoding catalase protein), a ctsb gene (encoding cathepsin B), a dhcr24 gene (encoding 24-dehydrocholesterol reductase), a gsr gene (encoding glutathione reductase), a gstk1 gene (encoding glutathione s-transferase kappa 1), a gstp1 gene (encoding glutathione s-transferase-1), an idh1 gene (encoding isocitrate dehydrogenase 1), an ncf1 gene (encoding neutrophil cytosolic factor 1 protein), one or more of the prdx1-4 genes (encoding peroxinedoxins 1-4), a prnp gene (encoding prion protein), a ptgs2 gene (encoding prostaglandin-endoperoxide synthase), an sic38a1 gene (encoding solute carrier family 38), a txn1 gene (encoding thioredoxin 1), a txnip gene (encoding thioredoxin interacting protein), a txnrd1 gene (encixling thioredoxin reductase 1), a txnrd2 gene (encoding thioredoxin reductase 2), or an ucp2 gene (encixling uncoupling protein 2). Alternatively or in addition, the evaluation may include a step of determining the concentration of 3-nitrotyrosine, measuring. The evaluation may include a step of determining the level of endogenous retrovirus Herv-K in (Lymphoma and ALS) and other environmentally activated endogenous retroviruses (e.g., retroviruses activated by oxidative stress).

In some embodiments, the evaluation may use a model system. The model system can be an animal model of disease, a cell culture system, an in vitro system, a mathematical model (e.g., a computational model), or a test carried out with a selected population of subjects (humans participating in a clinical trial).

Preventative Methods: As another respect, the present invention features a method for preventing a patient from developing a medical condition associated with brain dysfunction (or any of the particular conditions described herein). These methods include administering to the patient an amount of a composition described herein that is effective in reducing the likelihood that the patient will develop a given condition (as described herein).

It is to be understood that this invention is not limited to particular variations set forth and may of course, vary. Various changes may be made to the invention described and equivalents may be substituted without departing from the true spirit and scope of the invention. In addition, many mrxlifications may be made to adapt a particular situation, material, composition of matter, process, process act(s) or step(s). to the objective(s), spirit or scope of the present invention. All such modifications are intended to be within the scope of the claims made herein.

### EXAMPLES

We have quantified the efficacy of oral administration of different doses of (via oral gavage) for: (1) suppressing oxidative stress and inflammation and (2) stimulating the proliferation of hippocampal neural stem cells (NSCs) and increasing the extent of net neurogenesis in rats exposed to GWI-related (GWIR) chemicals and moderate levels of stress four months earlier (GWI-rats). We also aimed to identify an optimal dose of 5-amino-2,3-dihydro-1,4-phthalazinedione or the monosodium salt thereof that greatly regulates inflammation and oxidative stress and normalizes hippocampal neurogenesis in rats exposed to GWIR-chemicals and moderate levels of stress. In our studies neuronal fatigue was shown by determining the rate of mitochondrial recovery in the white blood cells after exercise.

Description of Experimental Procedures: The following narrative describes studies have been accomplished. The experiments comprises 5 major groups of rats:

Group 1: GWI-rats receiving 5-amino-2,3-dihydro-1,4-phthalazinedione at 40 mg/kg b.w.; Group 2: GWI-rats receiving 5-amino-2,3-dihydro-1,4-phthalazinedione at 80 mg/kg b.w.: Group 3: GWI-rats receiving 5-amino-2,3-dihydro-1,4-phthalazinedione at 160 mg/kg b.w.; Group 4: GWI-rats receiving vehicle (VEH); and Group 5: Age-matched naive control rats.

Animal numbers, survival and tissue harvesting: A total of 115 rats have been purchased so far in three different cohorts. The first cohort comprised 31 animals at the beginning of the study. Of those, 29 animals reached the endpoint of experiments; two animals were found dead during the four-month waiting period between the exposure of animals to GWIR chemicals and 15 minutes of restraint stress for 28 days and the commencement of 5-amino-2,3-dihydro-1,4-phthalazinedione treatment. The brain tissues from 29 animals were harvested for biochemical and molecular biological studies within these five groups: (1) 5-amino-2,3-dihydro-1,4-phthalazinedione, 40 mg/kg, n32 6; (2) 5-amino-2,3-dihydro-1,4-phthalazinedione, 80 mg/kg, n=6; (3) 5-amino-2,3-dihydro-1,4-phthalazinedione, 160 mg/kg, n=5; (4) GWI-vehicle (VEH), n=6; and (5) naive control, n=6. The second cohort comprised 42 animals at the beginning of the study. Of these 39 animals reached the endpoint of experiments. Two animals were found dead during the four-month waiting period between the exposure of animals to GWIR chemicals and stress and the commencement of 5-amino-2,3-dihydro-1,4-phthalazinedione treatment. An additional animal was euthanized in this period because it developed uncontrolled seizures, typified by continuous Stage-V seizures (bilateral forelimb clonus with rearing and filling). The brain tissues from 39 animals were harvested for immunohistochemical studies, within these five groups: (1) 5-amino-2,3-dihydro-1,4-phthalazinedione, 40 mg/kg, n=8; (2) 5-amino-2,3-dihydro-1,4-phthalazinedione, 80 mg/kg, n=8; (3) 5-amino-2,3-dihydro-1,4-phthalazinedione, 160 mg/kg, n=8; (4) GWI-VEH, n=8; and (5) naive control, n=7. The third cohort comprised 42 animals at the beginning of the study. Of these, 34 animals were exposed to GWIR-chemicals and stress and 8 animals were maintained in a control group. Animals exposed to GWIR-chemicals and stress were assigned to VEH or 5-amino-2,3-dihydro1,4-phthalazinedione treatment groups after they completed a 4-month waiting period.

Time-line of various procedures for animals in cohorts 1 and 2: Animals were exposed daily to GWIR-chemicals and stress for 28 days, and were subjected to a survival period of 4 months between exposure and treatment. The animals were then treated 5 times a week with 5-amino-2,3-dihydro-1,4-phthalazinedione or VEH for 8 weeks. 5′-bromodeoxyuridine (BrdU) is injected for 5 days in the 3rd week of treatment. The animals are then subjected to cognitive and mood function tests starting from the fifth week of treatment. Finally, euthanasia and tissue harvesting was performed after 8 weeks of treatment.

Exposure of animals to GWIR-chemicals and stress: Animals were exposed daily to the following chemicals for 28 days: pyridostigmine bromide (PB) at 2 mg/kg/day (via oral gavage), DEET at 60 mg/kg/day (via dermal application) and permethrin at 0.2 mg/kg/day (via dermal application). In addition, animals were subjected daily to 15 minutes of restraint stress using rat restrainers during the above 28-day period.

Survival period between exposure and treatment: Following the exposure to GMIR chemicals and stress, animals were maintained in the vivarium for four months in regular cages (two per cage) with ad libitum access to food and water. Administration of 5-amino-2,3-dihydro-1,4-phthalazinedione or VEH: Treatment was given for 8 weeks (5 times/week) via oral gavage, commencing in the 5th month after exposure to GMIR chemicals and stress. The doses of 5-amino-2,3-dihydro-1,4-phthalazinedione employed were 40 mg/kg, 80 mg/kg and 160 mg/kg.

BrdU injections: Subgroups of rats from all groups received BrdU injections in the 3rd week of drug/vehicle treatment daily for 5 days at a dose of 100 mg/kg/day.

Behavioral tests for assessing cognitive and mood function: We examined animals in all groups through stress-free behavioral tests. We used a Pattern Separation Test (PST) to assess cognitive function and a Sucrose Preference Test (SPT) to assess mood function. Euthanasia and tissue harvesting: Animals belonging to cohort I were deeply anesthetized with isoflurane in a small chamber until respiration ceased. Deeply anesthetized animals were decapitated following thoracotomyr and brain tissues were dissected rapidly for biochemical and molecular biological studies. Animals belonging to cohort 2 were first deeply anesthetized with isoflurane and then perfused through the heart with 4% parafbrmaldehyde solution. Fixed tissues were harvested for histological studies.

Analyses of oxidative stress: The hippocampal tissues obtained from animals belonging to cohort 1 were used for the following measurements. First, we analyzed the oxidative stress response and the expression of antioxidant genes using the Rat Oxidative Stress Response PCR Array from Qiagen. We analyzed the expression of 84 key genes involved in oxidative stress response and antioxidant activity in the hippocampus of animals belonging to different groups using quantitative real time PCR (qRT-PCR) to ascertain the effects of 5-amino-2,3-dihydro-1,4-phthalazinedione treatment on oxidative stress. Second, we measured lipid peroxidation through quantification of malondialdehyde. Lipid peroxides are unstable indicators of oxidative stress in cells that decompose to form more complex and reactive compounds such as malondialdehyde (MDA), a natural by-product of lipid peroxidation. Hence, we measured MDA in hippocampal tissue extracts from different groups of animals using a TBARS Assay Kit. Third, we quantified 3-nitrotyrosine. Increased modification of tyrosine residues in proteins to 3-nitrotyrosine by peroxynitrite or other potential nitrating agents is seen in tissues subjected to oxidative stress. Hence, we quantified 3-nitrotyrosine in hippocampal tissue extracts from different groups, using the nitrotyrosine ELISA Kit.

Analyses of inflammation: The hippocampal tissues obtained from animals belonging to cohort 1 were also used to measure the relative levels of inflammatory cytokines in different groups of animals. We employed the Rat Cytokine Plate Array from Signosis, which facilitated analyses of 16 rat cytokines in a high-throughput manner. The cytokines included: tumor necrosis factor-alpha (TNF-α), interleukin=1 alpha (IL-1α), interleukin-1 beta (IL-1β), vascular endothelial growth factor (VEGF), fibroblast growth factor beta (FGFβ), interferon gamma (IFNγ), interleukin=5 (IL-5), interleukin=6 (IL-6), interleukin=15 (IL-15), leptin, monocyte chemoattractant protein=1 (MCP-1), IFN-gamma-inducible protein 10 (IP-10 or CXCL10), stem cell factor (SCF), Regulated on Activation, Normal T Cell Expressed and Secreted (Rantes), Macrophage inflammatory protein 1 alpha (MIP-1α) and transforming growth factor-beta (TGFβ).

Immunohistochemical studies: Fixed brain tissues obtained from animals belonging to cohort 2 were processed for cryostat sectioning. Serial sections (every 15th or 20th) through the entire hippo campus arc currently being processed for immunohistochemical detection of BrdU+cells (i.e. newly born cells), doublecortin (DCX, a marker of newly born neurons), glial fibrillary acidic protein (GFAP, a marker of astrocytes), IBA-1 (a marker of all microglia) and ED-1 a marker of activated mieroglia. These multiple cell types will be quantified using stereology in the coming year (second year of the project).

Dose-response studies conducted so far using 5-amino-2,3-dihydro-1,4-phthalazinedione in GWI rats suggest the following.

5-amino-2,3-dihydro-1,4-phthalazinedione treatment at higher doses improves cognitive function in GWI rats: We examined the cognitive ability of GWI rats belonging to different groups (n=1114/group) through a pattern separation test (PST). Pattern separation function reflects proficiency for discriminating analogous experiences through storage of similar representations in a non-overlapping manner (Leutgeb et al., Science, 315:961-966, 2007; Yassa and Stark, Trends Neurosci., 34:515-525). In this test, each rat successively explored two different sets of identical objects (object types 1 and 2) placed on distinct types of floor patterns (Patterns 1 and 2 [P1 and P2]) for 5 minutes each in the two acquisition trials (separated by 30 minutes). Thirty minutes later, in the testing phase (Trial-3), each rat explored an object from trial 2 (which is now a familiar object) and an object from Trial-1 (which is now a novel object) placed on the floor pattern employed in trial 2 (P2).

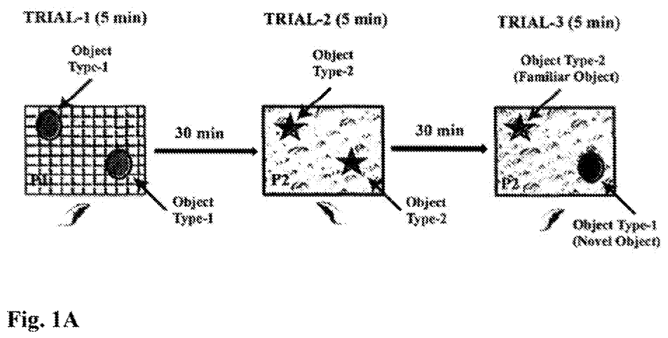

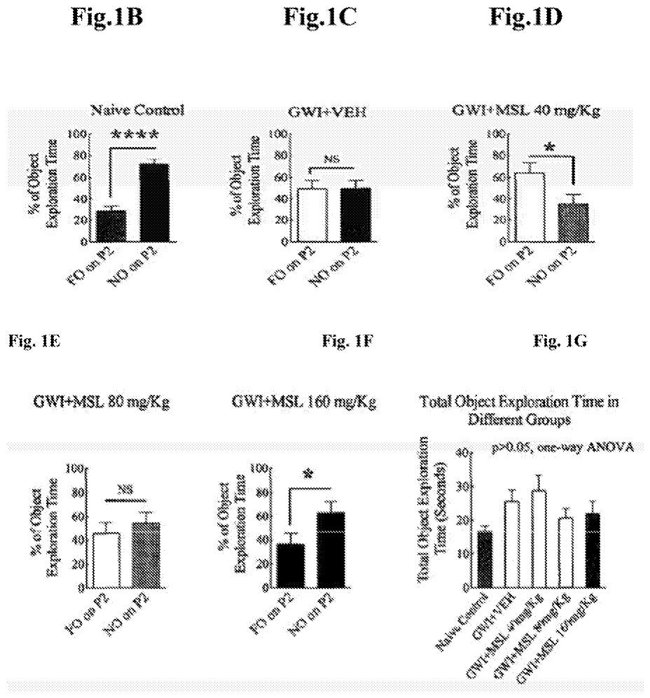

FIGS. 1A-1G illustrate the Pattern Separation Test (PST) and its results. FIG. 1A illustrates the sequence of trials, duration of trials, intervals between trials, examples of object types and floor patterns involved in this test. FIGS. 1B-1F compare percentages of object exploration time spent with the familiar object on pattern 2 (Fa on P2) and the novel object on pattern 2 (NO of P2) in different animal groups (n=11-14/group). Cyan, Naive control group; red, GWI+VEH group; orange, 5-amino-2,3-dihydro-1,4-phthalazinedione 40 mg/Kg group; yellow, GWI+5-amino-2,3-dihydro-1,4-phthalazinedione, 80 mg/Kg group; green, GWI+5-amino-2,3-dihydro-1,4-phthalazinedione, 160 mg/Kg group. FIG. 1G compares the total object exploration time between groups in Trial-3. one-way ANOVA analysis did not show ditierences between groups, implying that the specificity of the novel object exploration time (NO on P2) was not influenced by differences in the total object exploration time.

(i) Excellent pattern separation ability (i.e., ability to distinguish between similar experiences) in naive rats was revealed by a greater exploration of the object from trial 1 (i.e. novel object on pattern 2 [NO on P2]) than the object from trial 2 (i.e. familiar object on pattern 2 [FO on P2], p<0.0001, FIG. 1).

(ii) GWI rats that received VEH (GWI+VEH) showed no preference for the NO on P2, as they spent nearly similar amounts of time with novel and familiar objects on P2 (FIG. 1), implying loss of ability for pattern separation. Previous studies have shown that this task requires normal levels of dentate neurogenesis (Jain et al., PLoS One, 7:e46340, 2012; McAvoy et al., Front. Syst. Neurosci ., 9:120, eCollection, 2015; Oomen et al., Wiley Interdiscip. Rev. Cogn. Sci. 5:573-587, 2014). However, it remains to be examined whether these rats display decreased levels of dentate neurogenesis.

(iii) GWI rats that received lower doses of 5 5-amino-2,3-dihydro-1,4-phthalazinedione (40 mg/kg) remained impaired, which was evidenced by their greater exploration of the object from trial 2 (FO on P2]) than the object from trial 1 (NO on P2, p<0.05, FIG. 1), suggesting that a low dose of 5-amino-2,3-dihydro-1,4-phthalazinedione is not efficacious fir reversing pattern separation dysfunction. GWI rats that received moderate doses of 5-amino-2,3-dihydro-1,4-phthalazinedione (80 mg/kg) also remained impaired, as they spent similar amounts of time with novel and familiar objects on P2 (p>0.05, FIG. 1). In contrast, GWI rats that received higher doses of 5-amino-2,3-dihydro-1,4-phthalazinedione (160 mg/kg) displayed an ability for pattern separation. This was revealed by their greater exploration of the object from trial 1 (NO on P2) than the object from trial 2 (FO on P2), p<0.05, FIG. ). Taken together, this study suggested that cognitive impairment pertaining to pattern separation could be reversed through oral administration of relatively higher doses of 5-amino-2,3-dihydro-1,4-phthalazinedione in GWI rats. It has been shown that this cognitive improvement is related to increased levels of dentate neurogenesis in these rats, in comparison to GWI rats that received VEH during the same period.

5-amino-2,3-dihydro-1,4-phthalazinedione treatment at moderate to higher doses improves mood function in GWI rats: We examined mood function (or the extent of depressive-like behavior) in GWI rats belonging to different groups (n=11-14/group) through a Sucrose Preference Test (SPT), which is a stress free test measuring anhedonia (i.e. inability to tel pleasure, a measure of depression). This test comprised four days of monitoring. On day 1, rats were housed individually and given free access to two identical bottles containing 1% sucrose solution. Rats were trained to adapt to sucrose solution for 24 hours. On day 2, one bottle was replaced with a new bottle containing regular water for 24 hours. On day 3, rats were deprived of water and food for 23 hours, and then on day 4, rats were given free access to two bottles: one containing 100 ml of sucrose solution and another containing 100 ml of regular vyater. An hour later, the consumed volume in both bottles was recorded.

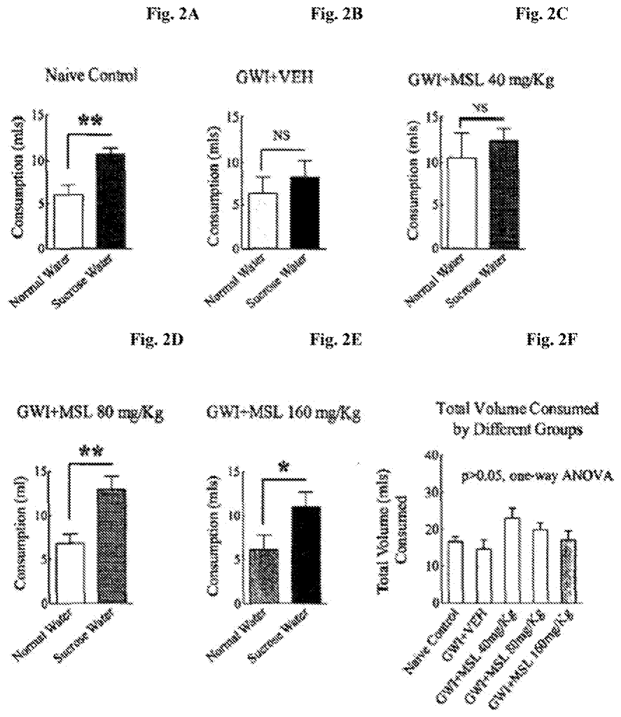

FIGS. 2A-2F illustrate the results of the Sucrose Preference Test (SPT). FIGS. 2A-2E compare the consumption of normal water and sucrose containing water in different animal groups (n=13-14/group in al GWI groups, n=6 in nave control group). Cyan, Naive control group; red, GWI+VEH group; orange, GWI+5-amino-2,3-dihydro-1,4-phthalazinedione (40 mg/kg) group; yellow, GWI+5-amino-2,3-dihydro-1,4-phthalazinedione (80 mg/kg) group; green, GWI+5-amino-2,3-dihydro-1,4-phthalazinedione (160 mg/kg) group.

FIG. 2F compares the total volume (normal water+sucrose-containing water) consumed by rats in each group. One-way ANOVA analysis did not show differences between groups, implying that the preference for drinking sucrose-containing water observed in naive control group and 5-amino-2,3-dihydro-1,4-phthalazinedione (80 mg/kg and 160 mg/kg groups) was not influenced by differences in the overall consumption of water during the testing period.

(i) Naive control rats clearly showed a preference for drinking sucrose-containing water over regular water (FIG. 2).

(ii) GWI rats that received VEH did not exhibit such preference as they consumed normal and sucrose-containing water in equal proportions (FIG. 2), implying the presence of anhedonia in GWI rats.

(iii) GWI rats that received lower doses of 5-amino-2,3-dihydro-1,4-phthalazinedione (40 mg/kg) also remained impaired, as they consumed sucrose-containing water and regular water in almost equal proportions (FIG. 2), suggesting that lower dose of 5-amino-2,3-dihydro-1,4-phthalazinedione does not have a positive effect on mood function in GWI rats.

(iv) GWI rats that received moderate and higher doses of 5-amino-2,3-dihydro-1,4-phthalazinedione (80 or 160 mg/kg) exhibited a clear preference for drinking sucrose-containing water over regular water (FIG. 2). Calculation of sucrose preference rate using the formula, sucrose consumption/water consumption+sucrose consumption)×100% also showed similar results (data not illustrated).

Thus, these results demonstrate that mood impairment, particularly anhedonia, may be reversed with oral administration of moderate to higher doses of 5-amino-2,3-dihydro-1,4-phthalazinedione in GWI rats. The study shows that improved mood with 5-amino-2,3-dihydro-1,4-phthalazinedione treatment is related to increased levels of dentate neurogenesis in these rats, in comparison to GWI rats that received VEH during the same period. This relationship has been examined quantitatively and in other studies we have been examining the effects of 5-amino-2,3-dihydro-1,4-phthalazinedione in several mood function tests.

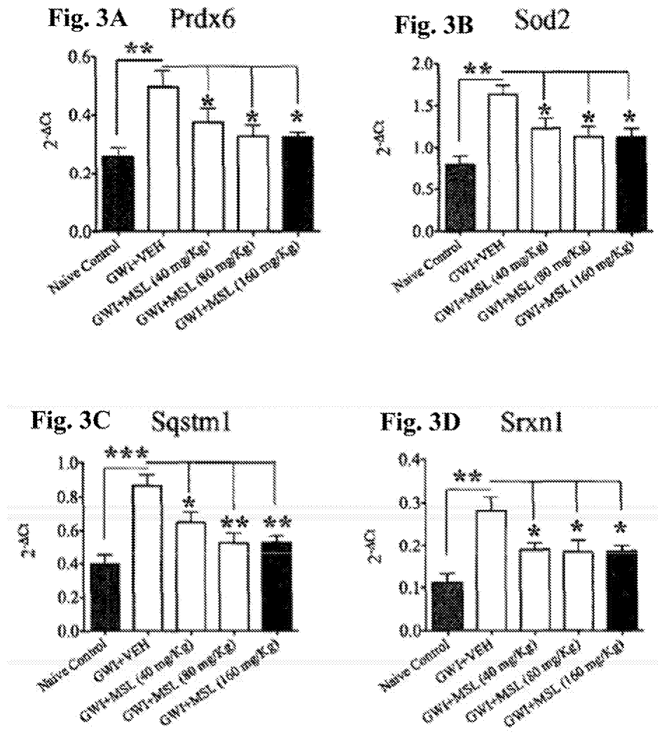

5-amino-2,3-dihydro-1,4-phthalazinedione treatment at higher doses modulates oxidative stress in the hippocampus of GWI rats: FIGS. 3A-3D demonstrate that 5-amino-2,3-dihydro-1,4-phthatazinedione treatment normalizes the expression of oxidative stress response genes prdx6, sod2,sqstm1 and srxn1 in GWI rats.

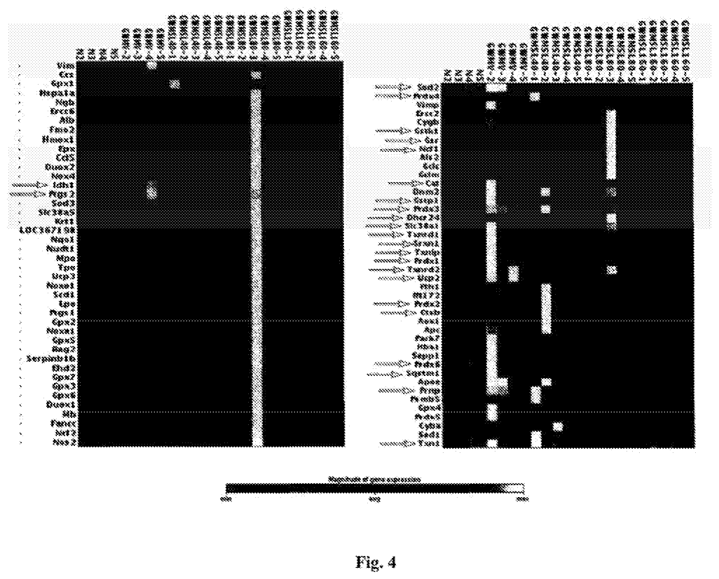

FIG. 4 is a clustergram showing the expression of oxidative stress response genes in various animal groups. N2-N5, naive control animals (n=4); GWMV2-GWMV5, GWI rats receiving vehicle (n=4); GWMSL40-1 to GWMSL40-5, GWI rats receiving 5-amino-2,3-dihydro-1,4-phthalazinedione at 40 mg/kg (n=5); GWMSL80-1 to GWMSL80-5, GWI rats receiving 5-amino-2,3-dihydro-1,4-phthalazinedione at 80 mg/kg (n=5):, GWMSL160-1 to GWMSL60-5, GWI rats receiving 5-amino-2,3-dihydro-1,4-phthalazinedione at 160 mg/kg (n=5). Arrows denote genes, which show upregulation GWI rats receiving VEH and normalization in GWI rats receiving 5-amino-23-dihydro-1,4-phthalazinedione particularly obvious in GWI rats receiving 80 mg/Kg or 160 mg/Kg doses. MSL is the monosodium salt of luminol(5-amino-2,3-dihydro-1,4-phthalazinedione or 5-amino-2,3-dihydrophthalazine-1,4-dione).

(i) Expression of oxidative stress response and antioxidant genes: We analyzed the expression of 84 key genes involved in oxidative stress response and antioxidant activity in the hippocampus of animals belonging to different groups using quantitative real time PCR (qRTPCR). Among 84 genes related to oxidative stress response examined in this experiment, GWIrats receiving VEH exhibited increased expression of 24 genes, in comparison to age-matched naive control animals (FIGS. 3 and 4), implying the presence of significant oxidative stress in the hippocampus GWI rats. Among these 24 genes, the expression of 4 genes was completely normalized by 5-amino-2,3-dihydro-1,4-phthalazinedione treatment (FIG. 3). This was evidenced through statistics (one-way ANOVA with Newman-Keuls multiple comparison test), which showed that GWI rats receiving 5-amino-2,3-dihydro-1,4-phthalazinedione exhibited reduced expression of these genes, in comparison to GWI rats receiving VEH. The genes comprise the following: (1) Prdx6: This gene encodes peroxiredoxin-6 protein. It is a member of the peroxiredoxin family of antioxidant enzymes (thiol-specific antioxidant protein family). It is involved in redox regulation of the cell, as it can reduce hydrogen peroxide and short chain organic, fatty acid, grid phospholipid hydroperoxides. It is also believed to play a role in the regulation of phospholipid turnover as well as in protection against oxidative injury; (2) Sod2: This gene encodes mitochondrial superoxide dismutase 2 protein. It is also known as manganese-dependent superoxide dismutase (MnSOD). Sod2 protein forms a homotetramer and binds one manganese ion per subunit. This protein binds to the superoxide byproducts of oxidative phosphorylation and converts them to hydrogen peroxide and diatomic oxygen, which facilitates SOD2 to clear mitochondrial reaction oxygen species (ROS) and thereby provides protection against cell death; (3) Sgstm1: This gene encodes sequestosome 1 (or p62) protein. This is a multitimetional protein that binds ubiquitin and regulates activation of the nuclear factor kappa-B (NF-kB) signaling pathway. The protein functions as a scaffolding/adaptor protein in concert with TNF receptor-associated factor 6 to mediate activation of NF-kB in response to upstream signals. Studies also suggest that this protein is a common component of protein aggregates that are found in protein aggregation diseases affecting the brain (e.g. Parkinson's and Alzheimer's diseases); and (4) Srxn1: This gene encodes sulfiredoxin-1 protein. This protein binds to peroxiredoxins and reduces over oxidized peroxiredoxins in the presence of cofactors including magnesium and ATP. Elevated expression of this protein has been associated with different types of malignant tumors. Thus, sulfiredoxin along with peroxiredoxins, play an important role in protecting tissues from oxidative stress.

Furthermore, 20 genes that displayed increased expression in. GWI rats were normalized to control levels by higher doses of 5-amino-2,3-dihydro-1,4-phthalazinedione treatment (80 or 160 mg/kg, FIG. 4). Their expression was greater in GWI rats receiving VEH than in naive control animals (p<0.05-0.01) but did tint differ from expression in GWI rats receiving higher doses of 5-amino-2,3-dihydro-1,4-phthalazinedione (p>0.05). Yet, as the overall reductions were moderate, their expression in GWI rats receiving 5-amino-2,3-dihydro-1,4-phthalazinedione did not differ statistically from GWI rats receiving VEH. The genes include a cat gene, encoding catalase protein, which is a key antioxidant enzyme that converts the reactive oxygen species hydrogen peroxide to water and oxygen; a Ctsb gene, encoding cathepsin B (also called as amyloid precursor protein secretase, which is a protein involved in the proteolytic processing of amyloid precursor protein; a Dhcr24 gene, encoding 24-dehydrocholesterol reductase, which is an oxidoreductase involved in cholesterol biosynthesis; a Gsr gene encoding glutathione reductase, which reduces oxidized glutathione disulfide to the sulthydryl form GSH, which is an important cellular antioxidant; a Gstk1 gene, encoding glutathione S-transferase kappa 1, which functions in cellular detoxification; (6) a Gstp1 encoding glutathione S-transferase-1, which plays an important role in detoxification by catalyzing the conjugation of many hydrophobic and elecuophilic compounds with reduced glutathione; an Idh1 gene, encoding isocitrate dehydrogenase 1, which converts isocitrate to 2-ketoglutarate to produce NADPH necessary for many cellular processes and protection against ROS; and an Ncf1 gene, encoding neutrophil cytosolic factor 1 protein, which is a 47 kDa cytosolic subunit of neutrophil NADPH oxidase (a multicomponent enzyme that is activated to produce superoxide anion); four Prdx1-4 genes, encoding peroxiredoxins 1-4, which are antioxidant enzymes involved in reducing hydrogen peroxide and alkyl hydroperoxides to water and alcohol with the use of reducing equivalents derived from thiol-containing donor molecules; a Prnp gene, encoding prion protein, which is a membrane glycosylphosphalidyl-inosiud-anchored glycoprotein that tends to aggregate into rod-like structures: a Ptgs2 gene encoding prostaglandin-endoperoxide synthase (also known as cyclooxygenase), which is a key enzyme in prostaglandin biosynthesis and acts both as a dioxygcnase and as a peroxidase; a SIc38a1 gene, encoding solute carrier family 38, member 1 protein, which is an important transporter of glutamine, an intermediate in the detoxification of ammonia and the production of urea; a Txn1 l gene, encoding thioredoxin 1, which participates in various redox reactions through the reversible oxidation of its active center dithiol to a disulfide and catalyzes dithioldisulfide exchange reactions; a Txnip gene, encoding thioredoxin interacting protein, which is believed to act as an oxidative stress mediator by inhibiting thioredoxin activity or by limiting its bioavailability; a Txnrd1 gene encoding thioredoxin reductase 1, which reduces thioredoxins as well as other substrates, and plays a role in selenium metabolism and protection against oxidative stress; a Txnrd2 gene, encoding thioredoxin reductase 2, which is a selenocysteine-containing flavoenzyme that maintains thioredoxins in a reduced state and thereby plays a key role in regulating the cellular redox environment; and a Ucp2 gene, encoding uncoupling protein 2 (mitochondrial, preton carrier), which separates oxidative phosphorylation from ATP synthesis with energy dissipated as heat, also referred to as the mitochondrial proton leak. This protein facilitates the transfer of anions from the inner to the outer mitochondrial membrane and the return transfer of protons from the outer to the inner mitochondrial membrane. one or more of these genes can be assessed in the methods of the present invention.

Taken together, our qRT-PCR analyses suggest that 5-amino-2,3-dihydro-1,4-phthalazinedione treatment considerably alleviates oxidative stress in GWI rats, which was evidenced through normalization of the expression of multiple genes (that are typically upregulated in conditions such as increased oxidative stress) to levels seen in naive control animals.

(i) Levels of malondialdehyde (a measure of lipid peroxidation): Lipid peroxides are unstable indicators of oxidative stress in cells that decompose to form more complex and reactive compounds such as maiondialdehyde (MDA), a natural by-product of lipid peroxidation. Hence, we measured MDA in hippocampal tissue extracts from different groups using TBARS Assay Kit. one-way ANOVA analyses showed significant differences between groups (p<0.01, F=5,6). Post-hoc analyses were done using Newman-Keuls multiple comparison test, which revealed the fallowing: findings: (1) MDA concentration in GWI rats receiving VEH was higher than naive control rats but the difference was not significant statistically; (2) GWI rats receiving lower dose of 5-amino-2,3-dihydro-1,4-phthalazinedione (40 mg/kg) displayed higher concentration of MDA than naive control rats (p<0.05), GWI rats receiving 80 mg/kg 5-amino-2,3-dihydro-1,4-phthalazinedione (p<0.05) and GWI rats receiving 160 mg/kg 5-amino-2,3-dihydro-1,4-phthalazinedione (p<0.01); and (3) GWI rats receiving higher dose of 5-amino-2,3-dihydro-1,4-phthalazinedione (160 mg/kg) displayed reduced MDA concentration than GWI rats receiving VEH. Thus, administration of a higher concentration of 5-amino-2,3-dihydro-1,4-phthalazinedione (160 mg/kg) decreases MDA concentration in GWI rats.

(ii) Concentration of 3-nitrotyrosine: Increased modification of tyrosine residues in proteins to 3-nitrotyrosine by peroxynitrite or other potential nitrating agents is seen in tissues subjected to oxidative stress. Hence, we quantified 3-nitrotyrosine in hippocampal tissue extracts from different groups using the nitrotvrosine ELISA Kit, However, 3-NT levels did not differ between groups (one-way ANOVA, p>0.05). Naive control animals and GWI rats that received VEH, and GWI rats that received 5-amino-2,3-dihydro-1,4-phthalazinedione exhibited similar levels of 3-NT (FIG. 4). Thus, 3-NT levels are not altered in the hippocampus of GWI rats.

Analyses of inflammation: The hippocampal tissues obtained from animals were used for measurement of the relative levels of inflammatory cytokines in different groups of animals. We employed “The Rat Cytokine Plate Array” from Signosis, which facilitated analyses of 16 rat cytokines in a high-throughput manner. The cytokines included: TNF-α, IL-1α, IL-1β, VEGF, FGFβ, , IFNγ, IL-5, IL-6, IL-15, leptin, MCP-1, IP-10 (or CXCL10), SCF, Rantes, MIP-1α and TGFβ. This study revealed no significant differences in the concentration of these cytokines between naive control animals, GWI rats receiving VEH, and GWI rats receiving different doses of 5-amino-2,3-dihydro-1,4-phthalazinedione (see Table 1 below).

TABLE 1

| | Naïve | GWI + | GWI + MSL | | | |
|---|---|---|---|---|---|---|
| | Control | VEH | 40 mg/Kg | GWI + | | One-way |
| Cytokine | Mean ± | Mean ± | Mean ± | MSL 160 | Mean ± | ANOVA |
| Measured | S.E.M. | S.E.M. | S.E.M. | mg/Kg | S.E.M. | P value |
| TNF-alpha | 0.15 ± 0.03 | 0.18 ± 0.02 | 0.15 ± 0.02 | 0.19 ± 0.03 | 0.18 ± 0.02 | p > 0.05 |
| IL-1 beta | 0.32 ± 0.07 | 0.40 ± 0.06 | 0.38 ± 0.05 | 0.42 ± 0.06 | 0.39 ± 0.06 | p > 0.05 |
| IFN-gamma | 0.19 ± 0.03 | 0.23 ± 0.03 | 0.19 ± 0.05 | 0.23 ± 0.03 | 0.28 ± 0.03 | p > 0.05 |
| Rantes (CCL5) | 0.14 ± 0.02 | 0.18 ± 0.02 | 0.15 ± 0.05 | 0.17 ± 0.03 | 0.18 ± 0.02 | p > 0.05 |
| MCP-1 | 0.12 ± 0.02 | 0.14 ± 0.02 | 0.09 ± 0.05 | 0.13 ± 0.02 | 0.10 ± 0.02 | p > 0.05 |
| IL-6 | 0.22 ± 0.03 | 0.26 ± 0.03 | 0.23 ± 0.05 | 0.28 ± 0.02 | 0.24 ± 0.03 | p > 0.05 |
| IL-1alpha | 0.15 ± 0.03 | 0.20 ± 0.03 | 0.17 ± 0.05 | 0.21 ± 0.03 | 0.17 ± 0.01 | p > 0.05 |
| SCF | 0.29 ± 0.06 | 0.36 ± 0.06 | 0.32 ± 0.05 | 0.36 ± 0.06 | 0.32 ± 0.07 | p > 0.05 |
| *MIP-1 alpha | 0.05 ± 0.02 | 0.13 ± 0.03 | 0.06 ± 0.05 | 0.07 ± 0.01 | 0.08 ± 0.02 | p > 0.05 |
| FGF-beta | 0.15 ± 0.03 | 0.21 ± 0.03 | 0.17 ± 0.05 | 0.21 ± 0.03 | 0.18 ± 0.03 | p > 0.05 |
| VEGF | 0.16 ± 0.04 | 0.22 ± 0.03 | 0.19 ± 0.05 | 0.22 ± 0.03 | 0.21 ± 0.03 | p > 0.05 |
| LEPTIN | 0.34 ± 0.09 | 0.46 ± 0.09 | 0.40 ± 0.05 | 0.50 ± 0.09 | 0.43 ± 0.11 | p > 0.05 |
| IL-5 | 0.17 ± 0.04 | 0.21 ± 0.04 | 0.19 ± 0.05 | 0.22 ± 0.03 | 0.19 ± 0.04 | p > 0.05 |
| IL-15 | 0.14 ± 0.03 | 0.13 ± 0.02 | 0.13 ± 0.05 | 0.14 ± 0.02 | 0.11 ± 0.02 | p > 0.05 |
| IL-10 | 0.086 ± 0.02 | 0.092 ± 0.02 | 0.085 ± 0.02 | 0.103 ± 0.02 | 0.086 ± 0.01 | p > 0.05 |

*MIP-1 alpha (macrophage inflammatory protein-1 alpha, a chemokine)
Naïve control versus GWI + VEH, p < 0.05 (two-tailed, unpaired t-test)

The only exception is MIP-1α, which showed increased expression in GWI rats receiving VEH, in comparison to naive control animals. This protein is produced by macrophages believed to be involved in inflammation and is typically upregulated in the brain in conditions such as Alzheimer's disease, multiple sclerosis and hypoxic-ischemic brain injury, increased expression of this chemokine is believed to enhance inflammation by attracting, more leucocytes to the brain parenchyma. Treatment with 5-amino-2,3-dihydro-1,4-phthalazinedione reduced the concentration of MIP-1α, although the decreases were not significant statistically.

What is claimed is:

1. Use of a compound of Formula I or a pharmaceutically acceptable composition comprising the compound of Formula I in the treatment of a medical condition associated with brain dysfunction: 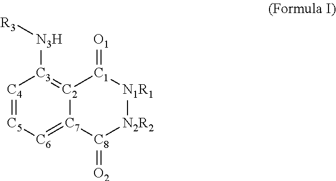
wherein, R₁ and R₂ are each, independently, hydrogen (H), lithium (Li), sodium (Na), potassitun (K), rubidium (Rb), caesium (Cs), or francium (Fr);
R₃ is an alkyl, alkenyl, alkynly, aryl, alkoxyl, alkenyloxy, alkynyloxy, aryloxy, alkoxy carbonyl, alkylamino, alkylthio, alkylsulfonyl, or alkylsulfinyl, each optionally substituted with an alkyl, halogen, alkoxy, aryl or heteroaryl moiety;
C₁, C₂, C₃, C₄, C₅, C₆, C₇ and C₈ are each, independently, carbon ¹² (¹²C) or an isotope of ¹²C (e.g., ¹³C);
N₁, N₂ and N₃ are each, independently, nitrogen 14 (¹⁴N) or an isotope of ¹⁴N (e.g., ¹⁵N); and O₁ and O₂ are each, independently, oxygen 16 (¹⁶O) or an isotope of ¹⁶O (e.g., ¹⁷O or ¹⁸O).

2. The use of claim 1, wherein the medical condition is a mood disorder or mood dysfunction, a memory disorder or memory dysfunction, anxiety or a stress-related condition, or an acute or chronic brain injury.

3. The use of claim 2, wherein the acute or chronic brain injury is caused by trauma, mitochondrial dysfunction, or dysfunction of an endogenous retrovirus.

4. The use of claim 2, wherein the mood disorder or mood dysfunction is bipolar disorder, depression (or a depressive illness), schizophrenia, or is associated with cancer, another chronic illness, infection, or substance abuse.

5. The use of claim 2, wherein the stress-related condition is a post-traumatic stress disorder.

6. The use of claim 1, wherein the medical condition is Gulf War Illness (GWI).

7. The use of claim 1, wherein the medical condition is, or has as a prominent symptom, amnesia, Alzheimer's disease (AD), dementia, Huntington's disease (HD), ataxia, Parkinson's disease (PD), Tourette's syndrome, migraine headache, multiple sclerosis (MS), multiple chemical sensitivity (MCS), cognitive dysfunction (CD), multiple sclerosis (MS), or amyotrophic lateral sclerosis (ALS).

8. The use of claim 1, wherein the compound of Formula I or the pharmaceutically acceptable composition comprising the compound of Formula I is formulated for oral administration or administration to a mucous membrane.

9. The use of claim 1, wherein the compound of formula I is 5-amino-2,3-dihydrophthalazine-1,4-dione (luminol), 6-amino-2,3-dihydrophthalazine-1,4-dione, 5-amino-2,3-dihydrophthalazine-1,4-dion-8-yl (luminyl), N-bromo-5-amino-2,3-dihydrophthalazine-1,4-dione, N -chloro-5-amino-2,3-dihydrophthalazine-1,4-dione, N-fluoro-5-a -dibydrophthalazine-1,4-dione, N-iodo-5-amino-2,3-dihydrophthalazine-1,4-dione, N-methyl-5-amino-2,3-dihydrophthalazine-1,4-dione, N -ethyl-5-amino-2,3-ditiydrophthaazine-1,4-dione, N-propyl-5-amino-2,3-dihydrophthalazine-1,4-dione, N-isopropyl-5-amino-2 ,3-dihydrophthalazine-4-dione, N-methanoyl-5-amino-2,3-dihydrophthalazine-1,4-dione, N-ethanoyl-5-amino-2,3-dihydrophthalazine-1,4-dione , N-propanoyl-5-amino-2 ,3-dihydrophthalazine-1,4-dione, N-hydroxyl-5-amino-2,3-dihydroptithalazine-1,4-dione, N-carboxyl-5-amino-2,3-dihydrophthalazine-1,4-dione, N -methanol-5-amino-2,3-dihydrophthalazine-1,4-dione, N-ethanol-5-amino-2,3-dihydrophthalazine-1,4-dione, N-propanol-5-amino-2,3-dihydrophthalazine-1,4-dione, N-methenyl-5-amino-2,3-dihydrophthalazine-1,4-dione, N-ethenyl -5-amino-2,3-dihydrophthalazine-1,4-dione, N-propenyl-5-amino-2,3-dihydrophthalazine-1,4-dione, N-methoxy-5-amino-2,3-dihydrophthalazine-1,4-dione N-etboxy-5-amino-2,3-dihydrophthalazine-1,4-dione, N-propoxy-5-amino-2,3-dihydrophthalazine-1,4-dione, N,N-dimethyl-5-amino-2,3-dihydrophthalazine-1,4-dione, N-acetylcysteine-5-amino-2,3-dihydrophthalazine-1,4-dione, N-acetylglutathione-5-amino-2,3-dihydrophthalazine-1,4-dione, 5-(hexanoyl oxyamino)-2,3-dihydrophthalazine-1,4-(methylamino)-2,3-dihydrophthalazine-1,4-dione, or 5-(acetoxyamino)-2,3-dihydrophthalazine-1,4-dione, a pharmaceutically acceptable salt thereof or an isotopic derivative thereof.

10. The use of claim 1, wherein the compound of formula I is:
monosodium 5-amino-2,3-dihydro-1,4-phthalazinedione;
monosodium 5-methamino-2,3-dihydro-1,4-phthalazinedione;
monosodium 5-acetoxyamino-2,3-dihydro-1,4-phthalazinedione; or
monosodium 5-(hexanoyl oxylamino)-2,3-dihydro-phthalazinedione.
